(19) United States
(12) Patent Application Publication
(10) Pub. No.: US 2024/0389532 A1
(43) Pub. Date: Nov. 28, 2024

(54) WEIGELA PLANTS

(71) Applicant: Spring Meadow Nursery, Inc., Grand Haven, MI (US)

(72) Inventor: Megan Mathey, Grand Haven, MI (US)

(21) Appl. No.: 18/200,196

(22) Filed: May 22, 2023

(51) Int. Cl. A01H 6/14; A01H 1/00

(52) U.S. Cl. CPC A01H 6/14; A01H 1/1245

(57) ABSTRACT

A Weigela plant having one or more of the following traits: 1) dark foliage color, 2) reblooming (remontant) (e.g., blooming multiple times throughout the growing season without pruning), 3) large flower size, 4) flowers that are bright pink, 5) petals that occasionally exhibit a medial stripe in a darker pink color, and 6) compact plant habit.

### FIELD OF INVENTION

The present disclosure relates to the field of Weigela plants, and more particularly toward the field of ornamental Weigela plants and methods of breeding the same.

### BACKGROUND

The genus Weigela belongs to the family Caprifoliaceae, otherwise known as the honeysuckle family, and is generally considered to be divided into around 12 species of deciduous shrubs. The Weigela is native to eastern Asia (China, Korea, and Japan).

Genetically, Weigela naturally occur as diploid (2n) plants with a genome size ranging from 1.91 to 2.32 pg. Only one triploid Weigela is known to exist in the commercial market as of 2022: Weigela ‘Courtalor’ CARNAVAL, developed by Duron and Decourtye utilizing colchicine treatment.

Weigela, specifically the florida species, established themselves as dependable flowering shrubs early in the landscape plant market. The genus is distinguishable by its rounded or mounding habit and profusions of brightly colored salverform flowers which emerge predominately in summer. Weigela are touted as being reliable flowering ornamental shrubs which are easy to maintain and vegetatively propagate. Their commercial value as ornamental plants have resulted in them becoming commonplace in home landscapes throughout the United States.

Although Weigela remain a frequent inclusion in a variety of landscape designs, more recent modern breeding endeavors have revitalized the genus by introducing greater variability in form as well as improved health and vigor. Greater interest has been directed towards developing Weigela with a broader range of foliage colors and habits, as well as encouraging increased flowing on compact and tidy habits.

Weigela are commonly implemented in landscape design and are therefore an important and high-value ornamental crop. As such, plant breeders of Weigela are continually seeking to develop marketable plants with novel characteristics, such as unique and dynamic foliage colors, extended bloom periods, and more vibrant flowers.

### SUMMARY

Plants of the new Weigela have not been observed under all possible combinations of environmental conditions and cultural practices. The phenotype may vary somewhat with variations in environmental conditions such as temperature and light intensity without, however, any variance in genotype.

The following traits have been repeatedly observed and are determined to be the unique characteristics of ‘SMNWFG’. These characteristics in combination distinguish ‘SMNWFG’ as a new and distinct Weigela plant:
1. Upright to outwardly spreading and uniformly mounding plant habit.
2. Vigorous growth habit and rapid growth rate.
3. Freely branching habit; dense and bushy appearance.
4. Leaves that are dark reddish brown in color with some green, generally along the midline.
5. Freely and remontant flowering habit.
6. Bright pink-colored flowers.
7. Good garden performance.

In one embodiment, a Weigela plant may include one or more of the physiological and morphological characteristics of the Weigela plant of variety ‘SMNWFG’. For instance, the Weigela plant variety may include one or more of the seven characteristics listed above. In one embodiment, a Weigela plant variety may include one or more of the following: 1) dark foliage color, 2) reblooming (remontant) (e.g., blooming multiple times throughout the growing season without pruning), 3) large flower size, 4) flowers that are bright pink, 5) petals that occasionally exhibit a medial stripe in a darker pink color, and 6) compact plant habit. In one embodiment, the Weigela plant variety may include remontancy in conjunction with a dark foliage trait.

In one embodiment, a method of producing F1 seed or embryo may involve providing a Weigela plant variety having one or more of the physiological and morphological characteristics of the Weigela plant of variety ‘SMNWFG’, including one or more of the following: 1) dark foliage color, 2) reblooming (remontant) (e.g., blooming multiple times throughout the growing season without pruning), 3) large flower size, 4) flowers that are bright pink, 5) petals that occasionally exhibit a medial stripe in a darker pink color, and 6) compact plant habit.

In one embodiment, a tissue or cell culture of regenerable cells produced from a Weigela of variety ‘SMNWFG’ is provided.

The foregoing and other embodiments can each optionally include one or more of the following features, alone or in combination. In particular, one embodiment includes all the following features in combination.

The tissue or cell culture may include tissues or cells from a plant part that include at least one of leaves, pollen, embryos, cotyledons, ovules, protoplasts, callus, pollen, seeds, petiole, hypocotyl, meristematic cells, roots, root tips, pistils, anthers, flowers, and stems.

A Weigela plant regenerated from the tissue or cell culture may be provided.

In one embodiment, a method of developing a Weigela plant variety having the physiological and morphological characteristics of a Weigela plant of variety ‘SMNWFG’ may be provided. The method may include genotyping a Weigela plant of variety ‘SMNWFG’, wherein the genotyping includes obtaining a sample of nucleic acids from the plant and detecting in the nucleic acids a plurality of polymorphisms, and using the identified polymorphisms for marker-assisted selection in a breeding program.

In one embodiment, a method for developing a Weigela plant variety may be provided. The method may include one or more of the following steps: a) identifying and selecting a spontaneous mutation of a Weigela plant of variety ‘SMNWFG’ or a part thereof, and cultivating the selected spontaneous mutation plant or plant part; b) introducing a mutation into the genome of a plant of variety ‘SMNWFG’ or a part thereof, and cultivating the mutated plant or plant part; and c) transforming a Weigela plant of variety ‘SMNWFG’ with a transgene.

The foregoing and other embodiments can each optionally include one or more of the following features, alone or in combination. In particular, one embodiment includes all the following features in combination.

A Weigela plant may be produced by cultivating the selected spontaneous mutation plant or plant part.

A Weigela plant may be produced by cultivating the mutated plant or plant part.

The mutation may be introduced using a method comprising one or more of the following: temperature, long-term seed storage, tissue culture conditions, ionizing radiation, chemical mutagens, targeting induced local lesions in genomes, zinc finger nuclease mediated mutagenesis, CRISPR/Cas9, meganucleases, and gene editing.

The method may include the transgene conferring resistance to an herbicide, insecticide, or disease. An herbicide, insecticide, or disease resistant plant may be produced by the method, which includes a transgene conferring resistance.

In one embodiment, a method of producing an F1 seed or embryo may be provided, where the method includes crossing a Weigela plant of variety ‘SMNWFG’ with a second plant and harvesting the resultant F1 seed or embryo.

The foregoing and other embodiments can each optionally include one or more of the following features, alone or in combination. In particular, one embodiment includes all the following features in combination.

The method of producing an F1 seed or embryo may include the second plant being another plant of variety ‘SMNWFG’.

The method of producing an F1 seed or embryo may include the second plant being a plant of a different variety from the variety ‘SMNWFG’.

The method of producing an F1 seed or embryo may include producing a Weigela plant by cultivating the harvested F1 seed or embryo.

In one embodiment, a method of producing an F1 seed or embryo may include providing a Weigela plant having one or more physiological and morphological characteristics of a Weigela plant of variety ‘SMNWFG’. The method may include crossing the Weigela plant with a second plant and harvesting the resultant F1 seed or embryo, where the one or more physiological and morphological characteristics of the Weigela ‘SMNWFG’ plant include at least one of remontancy and dark foliage.

The foregoing and other embodiments can each optionally include one or more of the following features, alone or in combination. In particular, one embodiment includes all the following features in combination.

The Weigela plant may be produced by cultivating the harvested F1 seed or embryo yielded according to the method of producing an F1 see or embryo.

In one embodiment, a Weigela plant variety may be provided having one or more physiological and morphological characteristics of a Weigela plant of variety ‘SMNWFG’, where the one or more physiological and morphological characteristics of the Weigela plant include at least one of dark foliage and remontancy.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components. Any reference to claim elements as “at least one of X, Y and Z” is meant to include any one of X, Y or Z individually, and any combination of X, Y and Z, for example, X, Y, Z; X, Y; X, Z; and Y, Z.

### BRIEF DESCRIPTION OF THE DRAWINGS

The patent or application file contains at least one drawing executed in color. Copies of this patent or patent application publication with color drawing(s) will be provided to the Office upon request and payment of the necessary fee.

The accompanying colored photographs illustrate the overall appearance of the new Weigela plant showing the colors as true as it is reasonably possible to obtain in colored reproductions of this type. Colors in the photographs may differ slightly from the color values cited in the detailed botanical description which accurately describe the colors of the new Weigela plant.

FIG. 1 shows plants of Weigela ‘SMNWFG’ grown outdoors in Grand Haven, Michigan.

FIG. 2 shows a plant of Weigela ‘SMNWFG’ grown outdoors.

FIG. 3 shows a close-up of flowers of a plant of Weigela ‘SMNWFG’.

FIG. 4 shows a close-up of flowers of a plant of Weigela ‘SMNWFG’.

FIG. 5 shows the pedigree of Weigela ‘SMNWFG’.

FIG. 6 shows a table of blooming periods in a greenhouse for a plant of Weigela ‘SMNWFG’ along with several conventional Weigela varietals.

FIG. 7 shows a table of blooming periods in a greenhouse for a plant of Weigela ‘SMNWFG’ along with several conventional Weigela varietals.

FIG. 8 shows a table of blooming periods in a field for a plant of Weigela ‘SMNWFG’ along with several conventional Weigela varietals.

FIG. 9 shows a table of blooming periods in a field for a plant of Weigela ‘SMNWFG’ along with several conventional Weigela varietals.

FIG. 10 shows a table of overall blooming periods for a plant of Weigela ‘SMNWFG’ along with several conventional Weigela varietals.

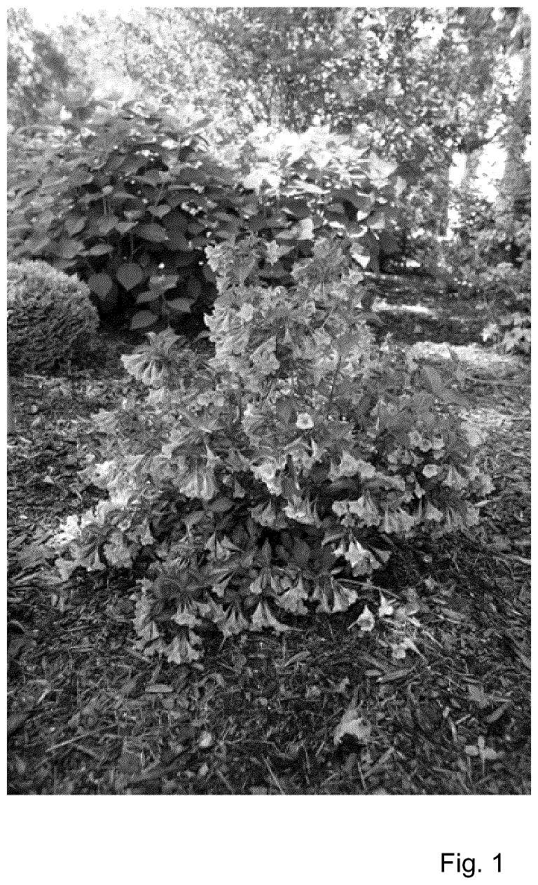

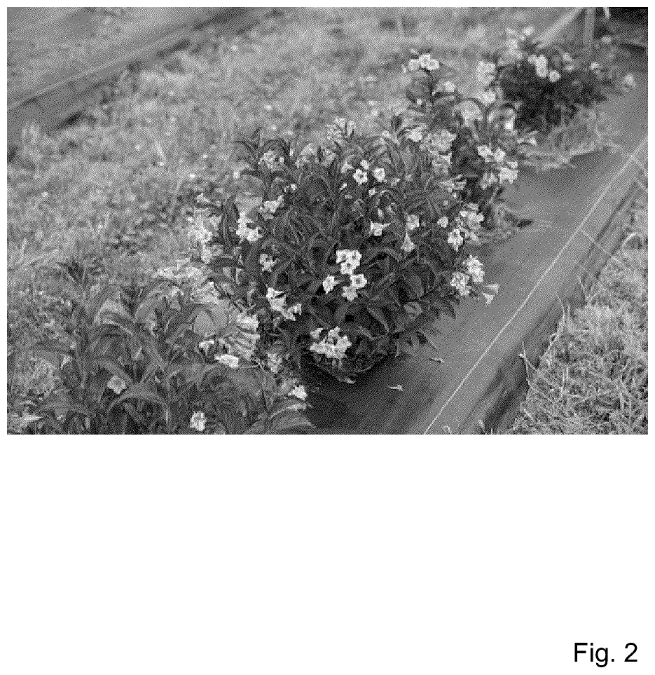

### Definitions

In the description and tables herein, a number of terms are used. In order to provide a clear and consistent understanding of the specification and claims, including the scope to be given such terms, the following definitions are provided:

Allele. “Allele” is any of one or more alternative forms for a gene.

Border Pattern. A “border pattern” refers to a pattern expressed on the flower where the color along the outer margin of the flower is different from the color of the rest of the flower. The color of the border along the outer margin of the flower may be thick or thin and solid or semi-solid. The color of the border along the outer margin and the rest of the flower may also vary in all colors and border patterns. Color, as referred to here, includes all pigmented colors and shades in-between, white, and unpigmented.

Gene. As used herein, “gene” refers to a segment of nucleic acid.

Locus. A “locus” is the position or location of a gene on a chromosome.

Plant Parts. A “plant part” or “a part thereof” is meant to refer to any part of the plant and includes but is not limited to, regenerable cells which may include plant calli, plant clumps, plant protoplast, plant cells, embryos, protoplasts, meristematic cells, callus, pollen, leaves, ovules, anthers, cotyledons, hypocotyl, pistils, roots, root tips, fruit, flowers, seeds, shoot, petiole, or stems.

Progeny. As used herein, the descendants of one or more of the parental lines and includes an F₁ Weigela plant produced from the cross of two Weigela plants where at least one plant includes a Weigela plant disclosed herein and progeny further includes, but is not limited to, subsequent F₂, F₃, F₄, F₅, F₆, F₇, F₈, F₉, and F₁₀ generational crosses with the recurrent parental line.

### DETAILED DESCRIPTION

All publications cited in this application are herein incorporated by reference in their entirety. The present disclosure relates to the field of ornamental Weigela plants and plant breeding. The disclosure provides new, distinct and stable Weigela plants with remontancy in conjunction with a dark foliage trade. This combination of traits has not been observed in any known Weigela, but was discovered through the Applicant's breeding program.

The SONIC BLOOM line of reblooming (remontant) Weigela florida possesses an assortment of flower color types (pink, pure pink, pearl, red). However, these Weigela cultivars all exhibit green foliage. The Weigela ‘SMNWFG’ is the first introduction to the SONIC BLOOM® line which exhibits dark (“wine-colored”) foliage. This foliage type is similar to the foliage exhibited by the classic “wine-colored” Weigelas (i.e., Weigela florida ‘Alexandra’ [WINE & ROSES®] US PP #10,772). However, the conventional “wine-colored” Weigelas currently available on the market do not exhibit the remontant trait of the Weigela ‘SMNWFG’ plant according to the present disclosure of the SONIC BLOOM line. The Weigela ‘SMNWFG’ is the first cultivar to include remontancy in conjunction with a dark foliage trait.

Weigela plants are considered to be valuable ornamental plants. Thus, continuing efforts of ornamental plant breeders are directed to developing plants with novel characteristics, such as flower color, growth habit, and foliage color. To accomplish this goal, the breeder may select and develop plants that have traits that result in superior Weigela varieties.

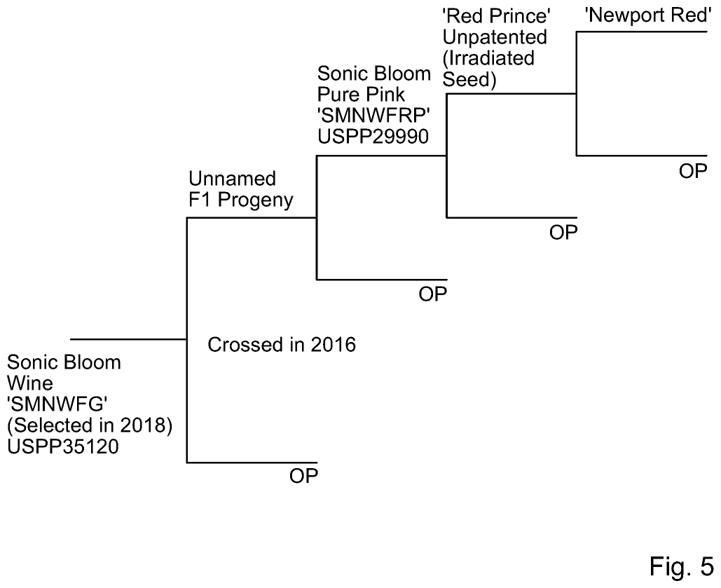

Weigela ‘SMNWFG’ was created by crossing and selection and is the result of four generations of controlled crossing, and is a hybrid varietal resulting from the crossing of a first varietal as the female, or seed, patent with a second unknown varietal of Weigela florida as a male, or pollen, parent by open pollination. The first varietal had been previously bred for target characteristics, and the Weigela ‘SMNWFG’ was selected within the progeny of the first and second identified varietals. The first varietal is an Unnamed F1 progeny of Weigela florida, which Applicant developed based on the Sonic Bloom Pure Pink ‘SMNWFRP’ variety (U.S. Plant Pat. 29,990) with another unknown varietal via open pollination. The Sonic Bloom Pure Pink ‘SMNWFRP’ varietal is based on irradiated seed obtained from the ‘Red Prince’ varietal and yet another unknown varietal via open pollination. The ‘Red Prince’ varietal is based on open pollination between the ‘Newport Red’ varietal and yet another unknown varietal. The pedigree for the Weigela ‘SMNWFG’ can be summarized by the lineage depicted in FIG. 5.

Asexual reproduction of the Weigela ‘SMNWFG’ plant by softwood stem cuttings has shown that the features of the Weigela ‘SMNWFG’ plant are stable and reproduced true to type in successive generations of asexual reproduction.

It has been found that the ‘SMNWFG’ variety described in the present application possesses the following characteristics:
1. Upright to outwardly spreading and uniformly mounding plant habit.
2. Vigorous growth habit and rapid growth rate.
3. Freely branching habit; dense and bushy appearance.
4. Leaves that are dark reddish brown in color with some green, generally along the midline.
5. Freely and remontant flowering habit.
6. Bright pink-colored flowers.
7. Good garden performance.

The ‘SMNWFG’ variety meets the needs of the horticultural industry. It can be grown to advantage as ornamentation in parks, gardens, public areas, and in residential settings. Accordingly, the Weigela ‘SMNWFG’ plant is particularly well suited for growing in the landscape.

The ‘SMNWFG’ variety of the present application can readily be distinguished from its ancestors. More specifically, plants of the Weigela ‘SMNWFG’ differ primarily from plants of the female parent selection in the following characteristics:
1. Leaves of plants of the Weigela ‘SMNWFG’ are dark reddish brown with some areas of green whereas leaves of plants of the female parent selection are dark green in color.
2. Plants of the Weigela ‘SMNWFG’ are more remontant than plants of the female parent selection.

Plants of the Weigela ‘SMNWFG’ can be compared to plants of the Weigela florida ‘SMWFDFPD’, disclosed in U.S. Plant Pat. No. 32,223. In comparing these plants to the Weigela ‘SMNWFG’, it can be seen that the Weigela ‘SMNWFG’ differ primarily from plants of ‘SMWFDFPD’ in the following characteristics:
1. Under high light conditions, leaves of plants of the Weigela ‘SMNWFG’ are not as heavily tinged as leaves of plants of ‘SMWFDFPD’.
2. Plants of the Weigela ‘SMNWFG’ are more remontant than plants of ‘SMWFDFPD’.

One embodiment of the Weigela ‘SMNWFG’ or a Weigela bred according to the breeding methods described herein, or both, may provide a compact plant habit. Compact plant habit is defined by a shortened internode length, dense growth, and a “full-looking” appearance in a container and in the landscape.

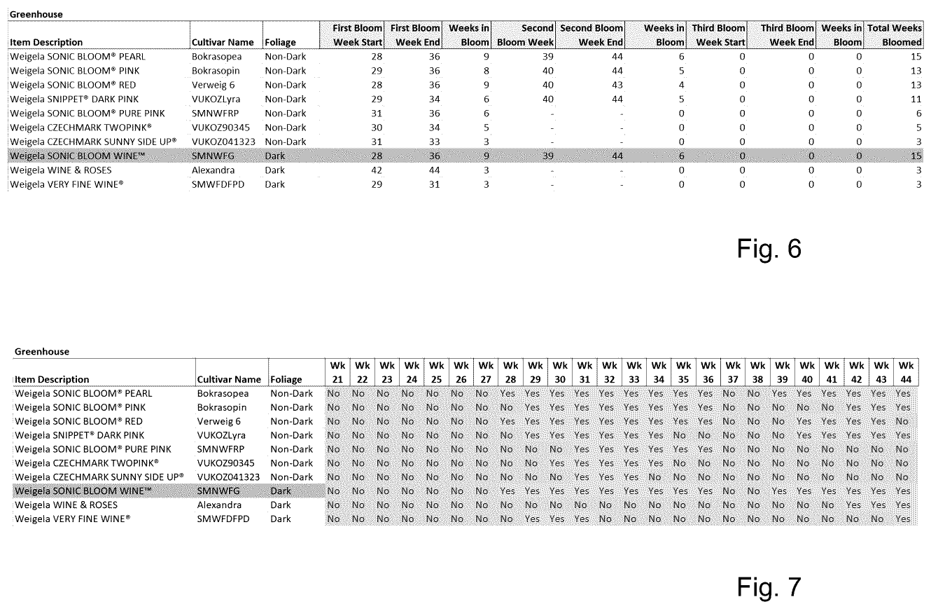

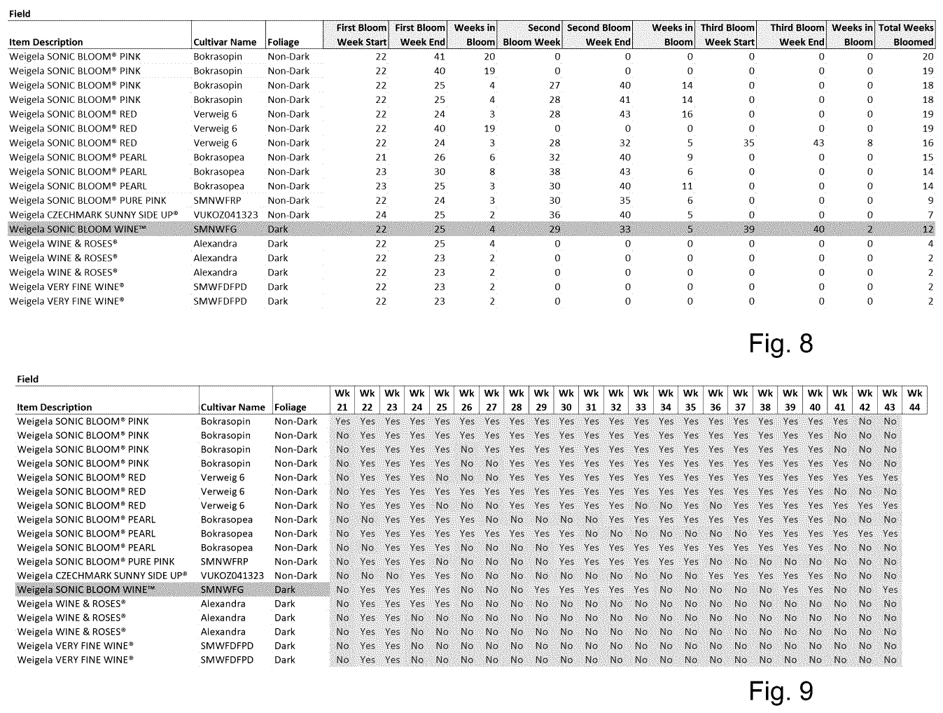

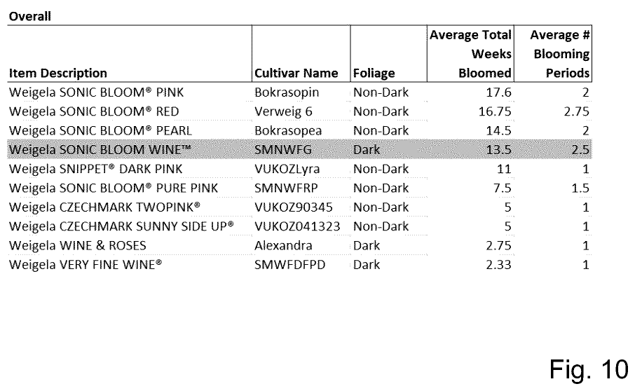

One embodiment of the ‘SMNWFG’ variety or a Weigela bred according to the breeding methods described herein, or both, is a reblooming trait (e.g., remontant) that involves blooming multiple times throughout the growing season without pruning in conjunction with dark foliage color. Dark foliage may be defined in contrast to the green foliage of the parent line. For instance, the dark foliage color may correspond to “wine-colored” foliage. Blooming data for the Weigela ‘SMNWFG’ demonstrating remontancy in conjunction with dark foliage can be seen in FIGS. 6-10 relative to several conventional Weigela plants. As can be seen in the provided tables, the Weigela ‘SMNWFG’ includes both dark foliage and remontancy absent from other Weigela varietals with dark foliage. This trait can be observed in both greenhouse and field settings, with first and second separate blooms being noticeable in the greenhouse, and with first, second, and third separate blooms being noticeable in the field. No other conventional Weigela appear to provide such a trait in conjunction with dark foliage. There are a number of conventional Weigela varietals identified in the provided tables that include remontancy, but these conventional Weigela varietals include non-dark foliage in contrast to the Weigela ‘SMNWFG’ of the present disclosure.

Yet another embodiment of the ‘SMNWFG’ variety or a Weigela bred according to the breeding methods described herein, or both, is large flower size. A conventional dark-foliaged Weigela such as ‘Alexandra’ [WINE & ROSES®] US PP #10,772) exhibits flowers which are around 2 cm in diameter on average. In contrast, the flowers of ‘SMNWFG’ are closer to 3 cm in diameter on average. The flower size of ‘SMNWFG’ is comparable to that of the other Weigela varieties in the SONIC BLOOM line, though none of these other plants possess the dark-foliage trait.

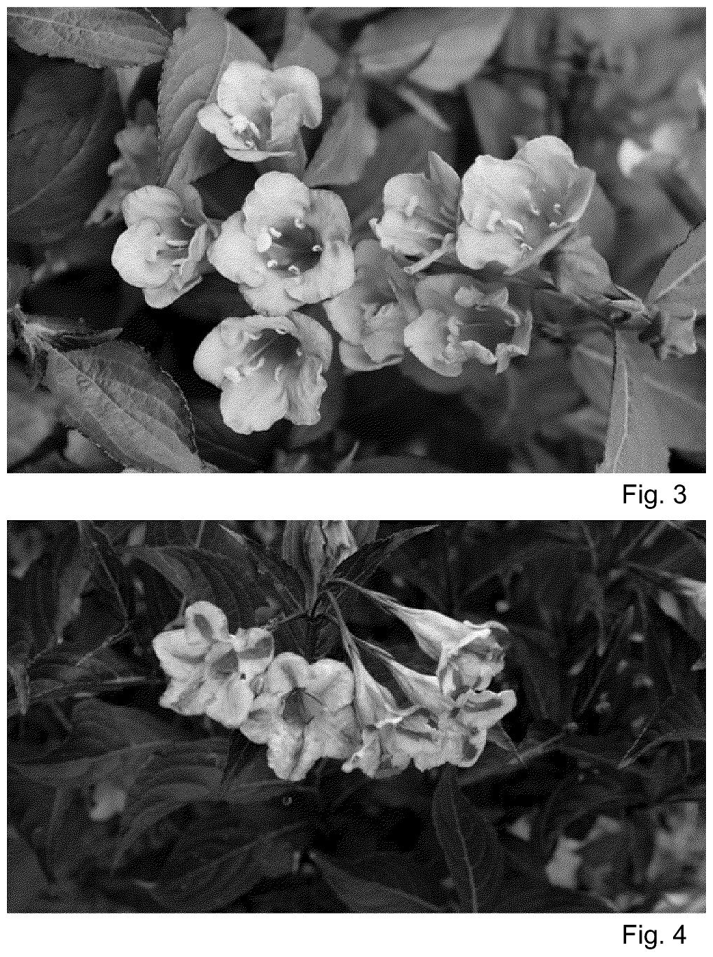

Another embodiment of the ‘SMNWFG’ variety or a Weigela bred according to the breeding methods described herein, or both, includes flowers that are bright pink. This trait can be seen in FIGS. 3 and 4.

Another embodiment of the ‘SMNWFG’ variety or a Weigela bred according to the breeding methods described herein, or both, includes petals that occasionally exhibit a medial strip in a darker pink color. This trait can be seen in FIG. 4.

It is to be understood that one or more embodiments according to the present disclosure may involve breeding, as described herein, to yield a Weigela plant having one or more traits of the ‘SMNWFG’ variety.

### I. Botanical Description

Plants used for the aforementioned photographs and the following description were grown during the spring in ground beds in an outdoor nursery and in three-gallon containers in a polypropylene-covered greenhouse in Grand Haven, Michigan, and under cultural practices typical of commercial Weigela production. During the production of the plants, day temperatures ranged from 18° C. to 27° C. and night temperatures ranged from 5° C. to 10° C. Plants of the Weigela were three years old when the photographs and description were taken. In the following description, color references are made to The Royal Horticultural Society Colour Chart, 2015 Edition, except where general terms of ordinary dictionary significance are used. The subject matter of the Weigela is described in U.S. patent application Ser. No. 17/953,103, filed Sep. 26, 2022, entitled Weigela Plant Named ‘SMNWFG’, and issued as U.S. Plant Pat. No. 35,120—the disclosure of which is hereby incorporated by reference in its entirety.

1. Botanical classification: Weigela florida ‘SMNWFG’
PROPAGATION
Type: By softwood stem cuttings.
Time to initiate roots, summer: About 15 days at temperatures about 18° C. to 27° C.
Time to produce a rooted young plant, summer: About two months at temperatures about 18° C. to 27° C.
Root Description: Fine, fibrous; typically white in color, actual color of the roots is dependent on substrate composition, water quality, fertilizer, substrate temperature and physiological age of roots.
Rooting Habit: Freely Branching; dense.

### Plant Description:

Plant and growth habit: Perennial shrub; upright to outwardly spreading and uniformly mounding plant habit; vigorous growth habit and rapid growth rate.
Branching Habit: Freely branching habit with about 30 lateral branches developing per plant; pinching enhances lateral branch development; dense and bushy plant form.
Plant height: About 60 cm.
Plant diameter: About 100 cm.
Lateral branch description: About 50 cm.
Diameter: About 5 mm.
Internode length: About 6 cm.
Texture: Slightly pubescent along longitudinal ridges; with development becoming less pubescent.
Strength: Strong.
Aspect: About 30° to 70° from vertical.
Color: Close to 144C tinged with close to 199A.

### Foliage

Leaf description:
   Arrangement: Opposite; simple.
   Length: About 5 cm to 7 cm.
   Width: About 2.5 cm to 3.5 cm.
   Shape: Elliptic.
   Apex: Acuminate.
   Base: Cuneate.
   Margin: Serrulate.
   Texture, upper surface: Slightly pubescent; coriaceous.
   Texture, lower surface: Slightly pubescence along venation; prominent venation; coriaceous.
   Venation pattern: Pinnate.
   Color: Developing leaves, upper and lower surfaces: Close to N144A tinged with close to 165A. Fully expanded leaves, upper and lower surfaces; Close to 147A tinged with close to 200A; venation, close to 144C; when exposed to high light conditions, upper surface will be more heavily tinged with dark reddish brown.
   Petioles: Length: About 4 mm. Diameter: About 3 mm.
   Texture, upper and lower surfaces: Somewhat pubescent.
   Color, upper surface: Close to 147A tinged with close to 200A.
   Color, lower surface: Close to 147A.

### Flower

Flower arrangement and habit: Salverform flowers arranged in terminal clusters or in small panicles each with about six to twelve flowers; about 100 to 150 flowers develop per lateral stem during the flowering season; flowers face upright to outwardly and drooping.
Fragrance: None detected.
Natural flowering season: Plants of the new Weigela flower repeatedly during the spring and summer in Grand Haven, Michigan; flowers not persistent.
Flower buds: Length: About 2.5 cm. Diameter: About 5 mm. Shape: Obovate to spatulate. Color: Close to 64B.
Flower diameter: About 2.75 cm.
Flower depth: About 3.5 cm.
Flower throat diameter: About 1.25 cm.
Flower tube length: About 3 cm.
Flower tube diameter, proximally: About 3 mm.
Corolla: Arrangement: Salverform; five petals fused into a tube with separate petal lobes.
Petal lobe length: About 1 cm.
Petal lobe width: About 1.25 cm.
Petal lobe shape: Roughly reniform.
Petal lobe apex: Obtuse, occasionally notched.
Petal lobe margin: Crenate; undulate and ruffled.
Petal texture, upper and lower surfaces: Smooth, glabrous; delicate.
Throat texture: Smooth, glabrous.
Tube texture: Glabrous, some creasing.
Color:
Developing petal lobes, upper surface: Close to 70C.
Developing petal lobes, lower surface: Close to 64B.
Fully expanded petal lobes, upper surface: Close to 64B, occasionally with a medial stripe, close to 64C; venation, close to 64B.
Fully expanded petal lobes, lower surface: Close to 64C; venation, close to 64C.
Throat: Close to 64C; venation, close to 64C.
Tube: Close to 64C; venation, close to 64C.
Sepals.—Quantity per flower: Five in a single whorl, fused at the base; calyx campanulate.
Length: About 1 cm.
Width: About 1 mm.
Shape: Narrowly triangular, elongated.
Apex: Acute.
Margin: Entire.
Texture, upper and lower surfaces: Smooth; glabrous.
Color, upper and lower surfaces: Close to 200A.
Peduncles.—Length: About 6 cm.
Diameter: About 3 mm.
Strength: Strong.
Aspect: About 90° from lateral branch axis.
Texture: Slightly pubescent.
Color: Close to 144C tinged with close to 199A.
Pedicels.—Length: About 2 cm.
Diameter: About 2 mm.
Strength: Strong; somewhat flexible.
Aspect: About 30° to 80° from peduncle axis.
Texture: Smooth, glabrous.
Color: Close to 200A.
Reproductive organs.—Stamens: Quantity and arrangement: Five per flower.
Filament length: About 1 cm.
Filament color: Close to 73B.
Anther shape: Narrowly oblong.
Anther size: About 5 mm.
Anther color: Close to 158B.
Pollen amount: Moderate.
Pollen color: Close to 158B.
Pistils: Quantity: One per flower.
Pistil length: About 3.5 cm.
Style length: About 3 cm.
Style color: Close to 73B.
Stigma shape: Peltate to globular, lobed.
Stigma color: Close to 158C.
Seeds and fruits: At the time of patent filing, seed and fruit data had not yet been recorded. This Weigela is not a non-fruiting variety.
Garden performance: Plants of the Weigela have exhibited good garden performance and to tolerate wind, rain and temperatures ranging from −31° C. to 38° C.
Pathogen & pest resistance: To date, plants of the Weigela ‘SMNWFG’ have not been observed to be resistant to pathogens and pests common to Weigela plants.

### II. Using Weigela Plants to Develop Other Weigela Plants

The Weigela plants herein, including the ‘SMNWFG’ variety, can also provide a source of breeding material that may be used to develop new Weigela plants and varieties. Plant breeding techniques known in the art and used in a Weigela plant breeding program include, but are not limited to, recurrent selection, mass selection, bulk selection, hybridization, mass selection, backcrossing, pedigree breeding, open-pollination breeding, restriction fragment length polymorphism enhanced selection, genetic marker enhanced selection, making double haploids, mutagenesis and transformation. Often combinations of these techniques may be implemented. There are many analytical methods available to evaluate a new variety. For instance, the observation of phenotypic traits or genotypic analysis, or both, may be used.

### III. Additional Breeding Methods

Any plants produced using the Weigela plants disclosed in the present application, including the ‘SMNWFG’ variety, and at least one parent are also an embodiment. Several methods of breeding the Weigela are described herein, but it is to be understood that the present disclosure is not so limited. Any type of breeding methodology may be implemented.

Descriptions of breeding methods can be found in one of several reference books (e.g., Allard, “Principles of Plant Breeding” (1999); Vainstein, “Breeding for Ornamentals: Classical and Molecular Approaches,” Kluwer Academic Publishers (2002); Callaway, “Breeding Ornamental Plants,” Timber Press (2000); and Bragdo, Marie, “Inter-specific Crosses in Lupinus: Cytology and Inheritance of Flower Color,” Institute of Genetics and Plant Breeding, Agricultural College of Norway, Vollebekk, Norway (Sep. 28, 1956)).

Breeding steps that may be used in the Weigela plant breeding program can include for example, pedigree breeding, backcrossing, mutation breeding, and recurrent selection. In conjunction with these steps, techniques such as RFLP-enhanced selection, genetic marker enhanced selection (for example, SSR markers), and the making of double haploids may be utilized.

As used herein, the term “plant” includes plant cells, plant protoplasts, plant cell tissue cultures from which Weigela plants can be regenerated, plant calli, plant clumps, and plant cells that are intact in plants or parts of plants, such as pollen, ovules, embryos, protoplasts, meristematic cells, callus, leaves, anthers, cotyledons, hypocotyl, pistils, roots, root tips, seeds, flowers, petiole, shoot, or stems and the like.

### A. Pedigree Breeding

Pedigree breeding starts with the crossing of two genotypes, such as a Weigela described herein, including the ‘SMNWFG’ variety, and another different Weigela plant having one or more desirable characteristics that is lacking or which complements a Weigela plant described herein. If the two original parents do not provide all the desired characteristics, other sources can be included in the breeding population. In the pedigree method, superior plants are selfed and selected in successive filial generations. In the succeeding filial generations, the heterozygous condition gives way to homogeneous varieties as a result of self-pollination and selection. In the pedigree method of breeding, five or more successive filial generations of selfing and selection may be practiced: F₁ to F₂; F₂ to F₃; F₃ to F₄; F₄ to F₅; etc. After a sufficient amount of inbreeding, successive filial generations may serve to increase seed of the developed variety. The developed variety may include homozygous alleles at about 95% or more of its loci.

### B. Backcross Breeding

Backcross breeding has been used to transfer genes for a simply inherited, highly heritable trait into a target homozygous variety or inbred line which is the recurrent parent. The source of the trait to be transferred is called the donor parent. After the initial cross, individuals possessing the phenotype of the donor parent are selected and repeatedly crossed (backcrossed) to the recurrent parent. The resulting plant is expected to have the attributes of the recurrent parent and the desirable trait transferred from the donor parent.

In addition to being used to create a backcross conversion, backcrossing can also be used in combination with pedigree breeding. As discussed previously, backcrossing can be used to transfer one or more target traits from one variety, the donor parent, to a developed variety called the recurrent parent, which has commercial characteristics of interest and yet lacks that target trait or traits. However, the same procedure can be used to move the progeny toward the genotype of the recurrent parent, but at the same time retain many components of the nonrecurrent parent by stopping the backcrossing at an early stage and proceeding with selfing and selection. For example, a Weigela plant may be crossed with another variety to produce a first-generation progeny plant. The first-generation progeny plant may then be backcrossed to one of its parent varieties to create a BC₁ or BC₂. Progeny are selfed and selected so that the newly developed variety has many of the attributes of the recurrent parent and yet several of the desired attributes of the nonrecurrent parent. This approach leverages the value and strengths of the recurrent parent for use in new Weigela varieties.

Therefore, another embodiment is a method of making a backcross conversion of a Weigela described herein, including the ‘SMNWFG’ variety, comprising the steps of crossing a Weigela with a donor plant comprising a desired trait, selecting an F₁ progeny plant comprising the desired trait, and backcrossing the selected F₁ progeny plant to a plant of Weigela described herein, including the ‘SMNWFG’ variety. This method may further comprise the step of obtaining a molecular marker profile of a Weigela described herein and using the molecular marker profile to select for a progeny plant with the desired trait and the molecular marker profile of a Weigela described herein.

### C. Recurrent Selection and Mass Selection

Recurrent selection is a method used in a plant breeding program to improve a population of plants. A Weigela described herein, including the ‘SMNWFG’ variety, may be suitable for use in a recurrent selection program. The method entails individual plants cross-pollinating with each other to form progeny. The progenies are grown and the superior progenies selected by any number of selection methods, which include an individual plant, half-sib progeny, full-sib progeny, and selfed progeny. The selected progenies are cross-pollinated with each other to form progeny for another population. This population is planted and again superior plants are selected to cross-pollinate with each other. Recurrent selection is a cyclical process and therefore can be repeated as many times as desired. The objective of recurrent selection is to improve the traits of a population. The improved population can then be used as a source of breeding material to obtain new varieties for commercial or breeding use, including the production of a synthetic variety. A synthetic variety is the resultant progeny formed by the intercrossing of several selected varieties.

Mass selection is a useful technique when used in conjunction with molecular marker enhanced selection. In mass selection, seeds from individuals are selected based on phenotype or genotype. These selected seeds are then bulked and used to grow the next generation. Bulk selection may involve growing a population of plants in a bulk plot, allowing the plants to self-pollinate, harvesting the seed in bulk, and then using a sample of the seed harvested in bulk to plant the next generation. Also, instead of self-pollination, directed pollination could be used as part of the breeding program.

Mass and recurrent selections can be used to improve populations of either self- or cross-pollinating plants. A genetically variable population of heterozygous individuals is either identified, or created, by intercrossing several different parents. The best plants, or plants satisfying target criteria, may be selected based on individual superiority, outstanding progeny, or excellent combining ability. The selected plants are intercrossed to produce a new population in which further cycles of selection are continued.

### D. Mutation Breeding

Mutation breeding is another method of introducing new traits into a Weigela plant according to one embodiment herein, including the ‘SMNWFG’ variety. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation; such as X-rays, Gamma rays (e.g., cobalt 60 or cesium 137), neutrons, (product of nuclear fission by uranium 235 in an atomic reactor), Beta radiation (emitted from radioisotopes such as phosphorus 32 or carbon 14), or ultraviolet radiation (preferably from 2500 to 2900 nm), or chemical mutagens (such as base analogues (5-bromo-uracil)), related compounds (8-ethoxy caffeine), antibiotics (streptonigrin), alkylating agents (sulfur mustards, nitrogen mustards, epoxides, ethylenamines, sulfates, sulfonates, sulfones, lactones), azide, hydroxylamine, nitrous acid, or acridines. Once a desired trait is observed through mutagenesis the trait may then be incorporated into existing germplasm by traditional breeding techniques. Details of mutation breeding can be found in Vainstein, “Breeding for Ornamentals: Classical and Molecular Approaches,” Kluwer Academic Publishers (2002). In addition, mutations created in other Weigela plants may be used to produce a backcross conversion of a Weigela plant described herein, including the ‘SMNWFG’ variety, that comprises such mutation.

Additional methods include, but are not limited to, expression vectors introduced into plant tissues using a direct gene transfer method, such as microprojectile-mediated delivery, DNA injection, electroporation, and the like. More preferably, expression vectors are introduced into plant tissues by using either microprojectile-mediated delivery with a biolistic device or by using Agrobacterium-mediated transformation. Transformant plants obtained with the protoplasm of the subject Weigela plants are intended to be within the scope of the embodiments of the application.

### E. Single-Gene Conversions

When the term Weigela plant is used in the context of an embodiment of the present application, this also includes any single gene conversions of a Weigela plant described herein. The term single gene converted plant as used herein refers to those Weigela plants which are developed by a plant breeding technique called backcrossing wherein all or nearly all of the target morphological and physiological characteristics of a variety are recovered in addition to the single gene transferred into the variety via the backcrossing technique. Backcrossing methods can be used with one embodiment of the present application to improve or introduce a characteristic into the variety. The term “backcrossing” as used herein refers to the repeated crossing of a hybrid progeny back to the recurrent parent, i.e., backcrossing 1, 2, 3, 4, 5, 6, 7, 8, or more times to the recurrent parent. The parental Weigela that contributes the gene for the target characteristic is termed the nonrecurrent or donor parent. This terminology refers to the fact that the nonrecurrent parent is used one time in the backcross protocol and therefore does not recur. The parental Weigela plant to which the gene or genes from the nonrecurrent parent are transferred is known as the recurrent parent as it is used for several rounds in the backcrossing protocol (Poehlman & Sleper (1994). In one backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a Weigela plant is obtained wherein all or nearly all of the target morphological and physiological characteristics of the recurrent parent are recovered in the converted plant, in addition to the single transferred gene from the nonrecurrent parent.

The selection of a suitable recurrent parent according to target criteria may provide for a successful backcrossing procedure. The goal of a backcross protocol is to alter or substitute a single trait or characteristic in the original variety. To accomplish this, a single gene of the recurrent variety is modified or substituted with the desired gene from the nonrecurrent parent, while retaining all or nearly all of the rest of the target genetics, and therefore the target physiological and morphological constitution of the original variety. The choice of the particular nonrecurrent parent may depend on the purpose of the backcross; one of the major purposes is to add some commercial trait or traits of interest to the plant. The exact backcrossing protocol may depend on the characteristic or trait being altered to determine an appropriate testing protocol. Although backcrossing methods may be simplified when the characteristic being transferred is a dominant allele, a recessive allele may also be transferred. In this instance, the breeding process may involve introduction of a test of the progeny to determine if the target characteristic has been successfully transferred.

Many single gene traits have been identified that are not regularly selected in the development of a new variety but that can be improved by backcrossing techniques.

F. Introduction of a New Trait or Locus into the Weigela Plant Described Herein

The Weigela plant described herein represent a new base of genetics into which a new locus or trait may be introgressed. Direct transformation and backcrossing represent two methods that can be used to accomplish such an introgression. The terms “backcross conversion” and “single locus conversion” are used interchangeably to designate the product of a backcrossing program.

### G. Transformation

Transformation methods include, but are not limited to, expression vectors introduced into plant tissues using a gene transfer method, such as microprojectile-mediated delivery, DNA injection, electroporation, and the like. In some embodiments, expression vectors are introduced into plant tissues of a Weigela described herein, including the ‘SMNWFG’ variety, by using either microprojectile-mediated delivery with a biolistic device or by using Agrobacterium-mediated transformation. Accordingly, further embodiments are methods of transformation using a Weigela plant described herein and the transformant plants obtained with the protoplasm of the subject Weigela plants.

### 1. Expression Vectors for ‘SMNWFG’ Transformation: Marker Genes

Plant transformation may involve the construction of an expression vector which will function in plant cells. Such expression vectors comprise DNA comprising a gene under control of, or operatively linked to, a regulatory element (e.g., a promoter). Expression vectors may include at least one genetic marker operably linked to a regulatory element that allows transformed cells containing the marker to be either recovered by negative selection (e.g., inhibiting growth of cells that do not contain the selectable marker gene) or by positive selection (e.g., screening for the product encoded by the genetic marker). Selectable marker genes used for plant transformation may include, for example, genes that code for enzymes that metabolically detoxify a selective chemical agent which may be an antibiotic or a herbicide, or genes that encode an altered target which is insensitive to the inhibitor. Positive selection methods may also be used.

One selectable marker gene used for plant transformation is the neomycin phosphotransferase II (nptII) gene which, when under the control of plant regulatory signals, confers resistance to kanamycin. Another selectable marker gene used for plant transformation is the hygromycin phosphotransferase gene which confers resistance to the antibiotic hygromycin.

Selectable marker genes for plant transformation not of bacterial origin include, for example, mouse dihydrofolate reductase, plant 5-enolpyruvylshikimate-3-phosphate synthase, and plant acetolactate synthase. See, e.g., Eichholtz, et al., SOMATIC CELL MOL. GENET., 13:67 (1987); Shah, et al., SCIENCE, 233:478 (1986); and Charest, et al., PLANT CELL REP., 8:643 (1990).

Another class of marker genes for plant transformation involves screening of presumptively transformed plant cells. Reporter genes are an example of this type of marker genes and can be used to quantify or visualize the spatial pattern of expression of a gene in specific tissues. Moreover, reporter genes can be fused to a gene or gene regulatory sequence for the investigation of gene expression. Marker genes used for screening presumptively transformed cells include β-glucuronidase (GUS), β-galactosidase, luciferase, and chloramphenicol acetyltransferase. See, e.g., Teeri, et al., EMBO J., 8:343 (1989); Koncz, et al., PROC. NATL. ACAD. SCI. USA, 84:131 (1987); and DeBlock, et al., EMBO J., 3:1681 (1984).

### 2. Expression Vectors for ‘SMNWFG’ Transformation: Promoters

Genes included in expression vectors must be driven by a nucleotide sequence comprising a regulatory element (e.g., a promoter). Many types of promoters may be used, as are other regulatory elements that can be used alone or in combination with promoters.

Some promoters are under developmental control and include promoters that preferentially initiate transcription in certain tissues, such as leaves, roots, seeds, fibers, xylem vessels, tracheids, or sclerenchyma. Such promoters may be referred to as “tissue-preferred” whereas promoters that initiate transcription only in a certain tissue may be referred to as “tissue-specific.” A “cell-type” specific promoter primarily drives expression in certain cell types in one or more organs, for example, vascular cells in roots or leaves. Whereas an “inducible” promoter may refers to a promoter which is under environmental control. Examples of environmental conditions that may affect transcription by inducible promoters include anaerobic conditions or the presence of light. Tissue-specific, tissue-preferred, cell-type specific, and inducible promoters constitute the class of “non-constitutive” promoters. A “constitutive” promoter refers to a promoter that is active under most environmental conditions. Many types of promoters may be used, such that it is to be understood the present disclosure is not limited to the promotors described and identified herein.

### 3. Additional Transformation Embodiments

The foregoing methods for transformation may be used for producing a transgenic variety. The transgenic variety could then be crossed with another (non-transformed or transformed) variety in order to produce a new transgenic variety. Alternatively, a genetic trait that has been engineered into a particular Weigela line using the foregoing transformation techniques could be moved into another line using other breeding techniques. For example, a backcrossing approach could be used to move an engineered trait from a publicly available variety into an elite variety, such as a Weigela plant described herein (including the ‘SMNWFG’ variety), or a backcrossing approach can be used to move a foreign gene from a variety containing the foreign gene in its genome into a variety that does not contain that gene.

Likewise, by means of such embodiments, genes of commercial interest can be expressed in transformed plants. More particularly, plants can be genetically engineered to express various phenotypes of commercial interest, including, but not limited to, genes that confer resistance to pests or disease, genes that confer resistance to an herbicide, genes that confer or contribute to a value-added or desired trait, genes that control male sterility, genes that create a site-for-site specific DNA integration, and genes that affect abiotic stress resistance. Many different genes are known and could potentially be introduced into a Weigela plant according to one embodiment described in the present disclosure. Non-limiting examples of particular genes and corresponding phenotypes one may choose to introduce into a Weigela plant include one or more genes for insect tolerance, such as a Bacillus thuringiensis (Bt.) gene, pest tolerance such as genes for fungal disease control, herbicide tolerance such as genes conferring glyphosate tolerance, and genes for quality improvements such as environmental or stress tolerances, or any desirable changes in plant physiology, growth, development, morphology, or plant product(s). For example, structural genes would include any gene that confers insect tolerance including but not limited to a Bacillus insect control protein gene as described in International Publication No. WO 99/31248, U.S. Pat. Nos. 5,689,052, 5,500,365 and 5,880,275. In another embodiment, the structural gene can confer tolerance to the herbicide glyphosate as conferred by genes including, but not limited to Agrobacterium strain CP4 glyphosate resistant EPSPS gene (aroA: CP4) as described in U.S. Pat. No. 5,633,435, or glyphosate oxidoreductase gene (GOX) as described in U.S. Pat. No. 5,463,175. Alternatively, the DNA coding sequences can affect these phenotypes by encoding a non-translatable RNA molecule that causes the targeted inhibition of expression of an endogenous gene, for example via antisense- or cosuppression-mediated mechanisms. The RNA could also be a catalytic RNA molecule (e.g., a ribozyme) engineered to cleave a desired endogenous mRNA product. See, e.g., Gibson and Shillito, MOL. BIOTECH., 7:125, 1997. Thus, any gene which produces a protein or mRNA which is necessary for a phenotype or morphology change of interest is useful for the practice of one or more embodiments described in the present disclosure.

### H. Backcross Conversions of Weigela Plant

A backcross conversion of the Weigela plant described herein occurs when DNA sequences are introduced through backcrossing (Allard, “Principles of Plant Breeding” (1999)) with the Weigela described herein being utilized as the recurrent parent. Both naturally occurring and transgenic DNA sequences may be introduced through backcrossing techniques. A backcross conversion may produce a plant with a trait or locus conversion in at least two or more backcrosses, including at least 2 crosses, at least 3 crosses, at least 4 crosses, at least 5 crosses, and the like. Molecular marker assisted breeding or selection may be utilized to reduce the number of backcrosses necessary to achieve the backcross conversion. For example, see, Openshaw, S. J., et al., Marker-assisted Selection in Backcross Breeding, Proceedings Symposium of the Analysis of Molecular Data, Crop Science Society of America, Corvallis, Oreg. (August 1994), where it is demonstrated that a backcross conversion can be made in as few as two backcrosses.

The complexity of the backcross conversion method depends on the type of trait being transferred (single genes or closely linked genes as compared to unlinked genes), the level of expression of the trait, the type of inheritance (cytoplasmic or nuclear), and the types of parents included in the cross. It is to be understood that for single gene traits that are relatively easy to classify, the backcross method is effective and manageable. See, Allard, “Principles of Plant Breeding” (1999). Target traits that may be transferred through backcross conversion include, but are not limited to, sterility (nuclear and cytoplasmic), fertility restoration, drought tolerance, nitrogen utilization, ornamental features, disease resistance (bacterial, fungal, or viral), insect resistance, and herbicide resistance. In addition, an introgression site itself, such as an FRT site, Lox site, or other site specific integration site, may be inserted by backcrossing and utilized for direct insertion of one or more genes of interest into a specific plant variety. In some embodiments, the number of loci that may be backcrossed into a Weigela plant described herein is at least 1, 2, 3, 4, or 5, and/or no more than 6, 5, 4, 3, or 2. A single locus may contain several transgenes, such as a transgene for disease resistance that, in the same expression vector, also contains a transgene for herbicide resistance. The gene for herbicide resistance may be used as a selectable marker and/or as a phenotypic trait. A single locus conversion of site specific integration system allows for the integration of multiple genes at the converted loci.

The backcross conversion may result from either the transfer of a dominant allele or a recessive allele. Selection of progeny containing the trait of interest is accomplished by direct selection for a trait associated with a dominant allele. Transgenes or genes transferred via backcrossing typically function as a dominant single gene trait and are relatively easy to classify. Selection of progeny for a trait that is transferred via a recessive allele requires growing and selfing the first backcross generation to determine which plants carry the recessive alleles. Recessive traits may involve additional progeny testing in successive backcross generations to determine the presence of the locus of interest. The last backcross generation may be selfed to give pure breeding progeny for the gene(s) being transferred, although a backcross conversion with a stably introgressed trait may also be maintained by further backcrossing to the recurrent parent with selection for the converted trait.

### I. Molecular Techniques Using Weigela Plant Described Herein

Molecular biological techniques may allow the isolation and characterization of genetic elements with specific functions. Molecular techniques include transformation. See Vainstein, “Breeding for Ornamentals: Classical and Molecular Approaches,” Kluwer Academic Publishers (2002).

1. Breeding with Molecular Markers

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select for the genome of the recurrent parent and against the genome of the donor parent. Using this procedure can minimize or reduce the amount of genome from the donor parent that remains in the selected plants. It can also be used to reduce the number of crosses back to the recurrent parent needed in a backcrossing program. The use of molecular markers in the selection process is often called genetic marker enhanced selection. Molecular markers may also be used to identify and exclude certain sources of germplasm as parental varieties or ancestors of a plant by providing a means of tracking genetic profiles through crosses. Molecular markers, which includes markers identified through the use of techniques such as Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs), and Single Nucleotide Polymorphisms (SNPs), may be used in plant breeding methods in connection with a Weigela plant described herein, including the ‘SMNWFG’ variety. See Vainstein, “Breeding for Ornamentals: Classical and Molecular Approaches,” Kluwer Academic Publishers (2002).

### 2. Genetic Marker Profile Through SSR and First Generation Progeny

In addition to phenotypic observations, a plant can also be identified by its genotype. The genotype of a plant can be characterized through a genetic marker profile which can identify plants of the same variety, or a related variety, or be used to determine or validate a pedigree. Genetic marker profiles can be obtained by techniques such as Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs) (which are also referred to as Microsatellites), and Single Nucleotide Polymorphisms (SNPs).

### 3. Signal Sequences for Targeting Proteins to Subcellular Compartments

Transport of a protein produced by a transgene to a sub cellular compartment, such as the chloroplast, vacuole, peroxisome, glyoxysome, cell wall, or mitochondrion, or for secretion into the apoplast, may be accomplished by means of operably linking a nucleotide sequence encoding a signal sequence (targeting sequence) typically to the 5′ and/or 3′ region of the transgene encoding the protein of interest. Signal sequences are well-known in the art. See, e.g., Becker, et al., PLANT MOL. BIOL., 20:49 (1992); Knox, et al., PLANT MOL. BIOL., 9:3-17 (1987); Lerner, et al., PLANT PHYSIOL., 91:124-129 (1989); Frontes, et al., PLANT CELL, 3:483-496 (1991); Matsuoka, et al., PROC. NATL. ACAD. SCI., 88:834 (1991); Gould, et al., J. CELL. BIOL., 108:1657 (1989); Creissen, et al., PLANT J., 2:129 (1991); Kalderon, et al., CELL, 39:499-509 (1984); and Steifel, et al., PLANT CELL, 2:785-793 (1990).

### 4. Gene Silencing

Techniques for gene silencing may be utilized, including, but not limited to, knock-outs (such as by insertion of a transposable element such as Mu) or other genetic elements such as a FRT, Lox, or other site specific integration sites; antisense technology; co-suppression; RNA interference; virus-induced gene silencing; target-RNA-specific ribozymes; hairpin structures; MicroRNA; ribozymes; oligonucleotide mediated targeted modification; Zn-finger targeted molecules; CRISPR/Cas9 system; and other methods or combinations of the above. See, e.g., Sheehy, et al., PNAS USA, 85:8805-8809 (1988); U.S. Pat. Nos. 5,107,065; 5,453,566; 5,759,829; Jorgensen, TRENDS BIOTECH., 8 (12): 340-344 (1990); Flavell, PNAS USA, 91:3490-3496 (1994); Neuhuber, et al., MOL. GEN. GENET., 244:230-241 (1994); Napoli, et al., PLANT CELL, 2:279-289 (1990); U.S. Pat. No. 5,034,323; Sharp, GENES DEV., 13:139-141 (1999); Zamore, et al., CELL, 101:25-33 (2000); Montgomery, et al., PNAS USA, 95:15502-15507 (1998); Burton, et al., PLANT CELL, 12:691-705 (2000); Baulcombe, CURR. OP. PLANT BIO., 2:109-113 (1999); Haseloff, et al., NATURE, 334:585-591 (1988); Smith, et al., NATURE, 407:319-320 (2000); U.S. Pat. Nos. 6,423,885; 7,138,565; 6,753,139; 7,713,715; Aukerman & Sakai, PLANT CELL, 15:2730-2741 (2003); Steinecke, et al., EMBO J., 11:1525 (1992); Perriman, et al., ANTISENSE RES. DEV., 3:253 (1993); U.S. Pat. Nos. 6,528,700; 6,911,575; 7,151,201; 6,453,242; 6,785,613; 7,177,766; 7,788,044; International Publication No. WO2014/068346; Martinelli, et al., Proposal of a Genome Editing System for Genetic Resistance to Tomato Spotted Wilt Virus 2014 AMERICAN JOURNAL OF APPLIED SCIENCES; Noman, et al., CRISPR-Cas9: Tool for Qualitative and Quantitative Plant Genome Editing, November 2016 FRONTIERS IN PLANT SCIENCE Vol. 7; and Zhang et al., Exploiting the CRISPR/Cas9 System for Targeted Genome Mutagenesis in Petunia February 2016 SCIENCE REPORTS Volume 6.

### J. Tissue Culture

Further reproduction of the variety can occur by tissue culture and regeneration. Tissue culture of various tissues of ornamental plants and a Weigela plant described herein and regeneration of plants therefrom may be utilized in breeding. For example, reproduction or breeding can be conducted according to one or more embodiments described in the following: Valla Rego, Luciana et al., Crop Breeding and Applied Technology. 1 (3): 283-300 (2001); Komatsuda, T., et al., Crop Sci., 31:333-337 (1991); Stephens, P. A., et al., Theor. Appl. Genet., 82:633-635 (1991); Komatsuda, T., et al., Plant Cell, Tissue and Organ Culture, 28:103-113 (1992); Dhir, S., et al., Plant Cell Reports, 11:285-289 (1992); Pandey, P., et al., Japan J. Breed., 42:1-5 (1992); and Shetty, K., et al., Plant Science, 81:245-251 (1992). Thus, another embodiment is to provide cells which upon growth and differentiation produce Weigela plants having one or more physiological and morphological characteristics of a Weigela plant described in the present application, including the ‘SMNWFG’ variety.

Regeneration refers to the development of a plant from tissue culture. The term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as pollen, ovules, embryos, protoplasts, meristematic cells, callus, leaves, anthers, cotyledons, hypocotyl, pistils, roots, root tips, flowers, seeds, petiole, shoot, or stems, and the like.

### K. Targeted Gene Editing

Targeted gene editing can be done using CRISPR/Cas9 technology (Saunders & Joung, Nature Biotechnology, 32, 347-355, 2014). CRISPR is a type of genome editing system that stands for Clustered Regularly Interspaced Short Palindromic Repeats. This system and CRISPR-associated (Cas) genes enable organisms, such as select bacteria and archaea, to respond to and eliminate invading genetic material. (Ishino, Y., et al. J. Bacteriol. 169, 5429-5433 (1987)). These repeats were known as early as the 1980s in E. coli, but Barrangou and colleagues demonstrated that S. thermophilus can acquire resistance against a bacteriophage by integrating a fragment of a genome of an infectious virus into its CRISPR locus. (Barrangou, R., et al. Science 315, 1709-1712 (2007)). Many plants have already been modified using the CRISPR system. See for example, U.S. Application Publication No. WO2014068346 (György et al., Identification of a Xanthomonas euvesicatoria resistance gene from pepper (Capsicum annuum) and method for generating plants with resistance), Martinelli, F. et al., “Proposal of a Genome Editing System for Genetic Resistance to Tomato Spotted Wilt Virus” American Journal of Applied Sciences 2014, and Noman, A. et al., and “CRISPR-Cas9: Tool for Qualitative and Quantitative Plant Genome Editing” Frontiers in Plant Science Vol. 7 Nov. 2016.

Gene editing can also be done using crRNA-guided surveillance systems for gene editing. Additional information about crRNA-guided surveillance complex systems for gene editing can be found in the following documents, which are incorporated by reference in their entirety: U.S. Application Publication No. 2010/0076057 (Sontheimer et al., Target DNA Interference with crRNA); U.S. Application Publication No. 2014/0179006 (Feng, CRISPR-CAS Component Systems, Methods, and Compositions for Sequence Manipulation); U.S. Application Publication No. 2014/0294773 (Brouns et al., Modified Cascade Ribonucleoproteins and Uses Thereof); Sorek et al., Annu. Rev. Biochem. 82:273-266, 2013; and Wang, S. et al., Plant Cell Rep (2015) 34:1473-1476. Therefore, it is another embodiment to use the CRISPR system on a Weigela plant of the instant application to, for example, modify traits and tolerances to pests and viruses.

Directional terms, such as “vertical,” “horizontal,” “top,” “bottom,” “upper,” “lower,” “inner,” “inwardly,” “outer” and “outwardly,” are used to assist in describing the invention based on the orientation of the embodiments shown in the illustrations. The use of directional terms should not be interpreted to limit the invention to any specific orientation(s).

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular.

### DEPOSIT INFORMATION

A deposit of Weigela plant ‘SMNWPG’ plant tissue disclosed herein will be made in accordance with all of the requirements of 37 C.F.R. §§ 1.801-1.809.

1. A tissue or cell culture of regenerable cells produced from a Weigela plant of variety ‘SMNWFG’, wherein a representative sample of plant tissue of said variety is to be deposited.

2. The tissue or cell culture of claim 1, comprising tissues or cells from a plant part that include at least one of leaves, pollen, embryos, cotyledons, ovules, protoplasts, callus, pollen, seeds, petiole, hypocotyl, meristematic cells, roots, root tips, pistils, anthers, flowers, and stems.

3. A Weigela plant regenerated from the tissue or cell culture of claim 1.

4. A method of developing a Weigela plant variety having the physiological and morphological characteristics of a Weigela plant of variety ‘SMNWFG’, wherein a representative sample of plant tissue of said variety is to be deposited, said method comprising:
genotyping a Weigela plant of variety ‘SMNWFG’, wherein said genotyping includes obtaining a sample of nucleic acids from the plant and detecting in the nucleic acids a plurality of polymorphisms, and using the identified polymorphisms for marker-assisted selection in a breeding program.

5. A method for developing a Weigela plant variety, comprising one or more of:
a) identifying and selecting a spontaneous mutation of a Weigela plant of variety ‘SMNWFG’ or a part thereof, and cultivating the selected spontaneous mutation plant or plant part;
b) introducing a mutation into the genome of a plant of variety ‘SMNWFG’ or a part thereof, and cultivating said mutated plant or plant part; or
c) transforming a Weigela plant of variety ‘SMNWFG’ with a transgene;
wherein a representative sample of plant tissue of the variety ‘SMNWFG’ is to be deposited.

6. A Weigela plant produced by cultivating the selected spontaneous mutation plant or plant part of claim 5.

7. A Weigela plant produced by cultivating said mutated plant or plant part of claim 5.

8. The method of claim 5, wherein the mutation is introduced using a method comprising one or more of the following: temperature, long-term seed storage, tissue culture conditions, ionizing radiation, chemical mutagens, targeting induced local lesions in genomes, zinc finger nuclease mediated mutagenesis, CRISPR/Cas9, meganucleases, and gene editing.

9. The method of claim 5, wherein the transgene confers resistance to a herbicide, insecticide, or disease.

10. A herbicide, insecticide, or disease resistant plant produced by the method of claim 9.

11. A method of producing an F1 seed or embryo, wherein the method comprises crossing a Weigela plant of variety ‘SMNWFG’ with a second plant and harvesting the resultant F1 seed or embryo,
wherein a representative sample of plant tissue of the variety ‘SMNWFG’ is to be deposited.

12. The method of claim 11, wherein the second plant includes another plant of variety ‘SMNWFG’.

13. The method of claim 11, wherein the second plant is a plant of a different variety from the variety ‘SMNWFG’.

14. A Weigela plant produced by cultivating the harvested F1 seed or embryo of claim 11.

15. A method of producing an F1 seed or embryo, the method comprising:
providing a Weigela plant having one or more physiological and morphological characteristics of a Weigela plant of variety ‘SMNWFG’, wherein a representative sample of plant tissue of the variety ‘SMNWFG’ is to be deposited; and
crossing the Weigela plant with a second plant and harvesting the resultant F1 seed or embryo, wherein the one or more physiological and morphological characteristics of the Weigela plant include remontancy and dark foliage.

16. A Weigela plant produced by cultivating the harvested F1 seed or embryo of claim 15.

17. A Weigela plant variety comprising:
one or more physiological and morphological characteristics of a Weigela plant of variety ‘SMNWFG’, wherein a representative sample of plant tissue of the variety ‘SMNWFG’ is to be deposited, wherein the one or more physiological and morphological characteristics of the Weigela plant include remontancy and dark foliage.
